SEQUENCE LISTING (1 sequence)

SEQ ID NO: 1 - DNA, 405 bp, Artificial Sequence
  mutated MS2 virus coat protein
catatggccagcaactttacccagttcgttctggttgacaacggcggttagggcgacgtgacggtggcgccgtctaacttcgccaacggtgttgcggagtggatttcttctaattctcgcagccaagcctataaagttacgtgttctgtgcgtcagtctagcgcccagaatcgcaagtacaccattaaagtggaggtgccgaaggtggcgacgcaaaccgtgggtggtgtggagctgccagttgcggcctggcgtagctatctgaacatggagctgacgatcccaatctttgccaccaatagcgactgcgaactgattgtgaaggccatgcagggtctgctgaaggatggtaacccgattccatctgccattgccgcgaactctggtatctactaataagtcgac

(19) United States
(12) Patent Application Publication
(10) Pub. No.: US 2010/0168402 A1
(43) Pub. Date: Jul. 1, 2010

(54) Direct Attachment of Polypeptides to Virus Like Particles

(76) Inventors: Bradley C. Bundy, Mountain View, CA (US); James R. Swartz, Menlo Park, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/619,442

(22) Filed: Nov. 16, 2009

Related U.S. Application Data
(60) Provisional application No. 61/199,240, filed on Nov. 14, 2008.

(51) Int. Cl. C07K 14/005

(52) U.S. Cl. 530/402

(57) ABSTRACT

Compositions and methods are provided for the control of direct protein attachment to virus like particles where virus structural proteins that have been modified to comprise an unnatural amino acid at a pre-determined site are reacted with one or more “display” polypeptides that also comprise an unnatural amino acid at a pre-determined site in a one step reaction. The compositions of the invention are useful for various purposes where it is desirable to efficiently and directly attach multiple polypeptides to a single carrier entity, particularly where two or more different polypeptides are attached to a single carrier.

### BACKGROUND OF THE INVENTION

Virus like particles (VLPs) consist of viral proteins derived from the structural proteins of a virus, usually in the absence of a viral genome. VLPs have received considerable attention for vaccines, targeted drug delivery, targeted gene delivery, and nanotechnology applications. VLP vaccines that are currently approved by the Food and Drug Administration (FDA) include human papillomavirus and Hepatitis B vaccines, which are very effective at eliciting both T cell and B cell immune responses.

The vast majority of eukaryote-infecting-virus-based VLPs have been synthesized using the insect-cell-based baculovirus expression system or mammalian-cell-based protein expression systems. Although the synthesis of virus-like particles has been attempted in cell-free systems, yields have been extremely low in eukaryotic cell-free systems (Lingappa et al. 2005. Virology 333:114), and assembly has failed in conventional prokaryotic systems (Katanaev et al. 1996. FEBS 397:143).

Virus-like particle (VLP) vaccines are typically comprised of multiple copies of a protein that, when assembled together, mimic the conformation of a native virus. In the currently approved vaccines, the virus coat proteins themselves are the antigen of interest, and thus the virus protein is derived from the pathogen of interest. However, there has also been interest in using a VLP as a carrier for heterologous antigens, e.g. polypeptide antigens.

Carrico et al. (2008) Chem Commun 10:1205-1207 demonstrated the site specific incorporation of para-amino-L-phenylalanine in the MS2 coat protein VLP. Short peptides of 7 to 15 amino acids in length were synthesized to include an N-terminal N,N-diethyl-N′ acylphenylene diamine. They were then individually attached to the VLP.

Strable et al. (2008) Bioconjugate Chem 19:866-875 substituted all methionines of a viral coat protein with the unnatural amino acids azidohomoalanine and homopropargyl glycine, and linked a heterologous protein through a linker to the azide group. However, the complete substitution of all methionines results in proteins with multiple unnatural amino acids.

Methods for the direct and highly efficient linkage of one or more heterologous polypeptides are of great interest for the use of VLPs as carriers. The present invention addresses this need.

### RELEVANT LITERATURE

Calhoun and Swartz (2005) Biotechnol Bioeng 90(5):606-13; Jewett and Swartz (2004) Biotechnol Bioeng 86(1):19-26; Jewett et al. (2002) Prokaryotic Systems for In Vitro Expression. In: Weiner M, Lu Q, editors. Gene cloning and expression technologies. Westborough, Mass.: Eaton Publishing. p 391-411; Lin et al. (2005) Biotechnol Bioeng 89(2):148-56.

U.S. Pat. No. 6,593,103. Noad and Roy (2003) Virus-like particles as immunogens. Trends in Microbiology 11(9):438-444. Palucha et al. 2005. Virus-like particles: models for assembly studies and foreign epitope carriers. Progress in Nucleic Acid Research and Molecular Biology 80:135-168. Spirin et al. 1988. A continuous cell-free translation system capable of producing polypeptides in high yield. Science 242:1162-1164.

International application U.S.07/15270; International application U.S.07/15170.

U.S. Pat. No. 6,337,191 B1; Swartz et al. U.S. Patent Published Application 20040209321; Swartz et al. International Published Application WO 2004/016778; Swartz et al. U.S. Patent Published Application 2005-0054032-A1; Swartz et al. U.S. Patent Published Application 2005-0054044-A1; Swartz et al. International Published Application WO 2005/052117. Bachmann and Jennings (2004). Virus-like particles: Combining innate and adaptive immunity for effective vaccination. In: Kaufmann SHE, editor. Novel Vaccination Strategies. Weinheim: WILEY-VCH Verlag GmbH & Co. KGaA. p 415-430.

Brown et al. (2002) Intervirology 45(6):371-380. Grgacic and Anderson (2006) Virus-like particles: Passport to immune recognition. Methods 40(1):60-65. Jegerlehner et al. (2002) Vaccine 20(25):3104-3112; Kratz et al. (1999) Proc Natl Acad Sci USA 96:1915-1920; Wu et al. (1995) Bioconjugate Chem 6:587-595.

### SUMMARY OF THE INVENTION

Compositions and methods are provided for the control of direct protein attachment to virus like particles. In the methods of the invention, virus structural proteins that have been modified to comprise an unnatural amino acid at a pre-determined site and that have assembled into a carrier VLP are reacted in a single step reaction with one or more “display” polypeptides that also comprise an unnatural amino acid at a pre-determined site, where the unnatural amino acids form a stable bond. The unnatural amino acid on the virus structural protein, or “carrier”, is different from, and reactive with, the unnatural amino acid present on the display polypeptide(s). In some embodiments of the invention, one of the unnatural amino acids comprises an azide moiety, for example p-azido-L-phenylalanine; and the other unnatural amino acid comprises a terminal alkyne, for example propargyloxyphenylalanine. In the presence of a copper catalyst the azide and alkyne moieties react to generate a stable triazole linkage.

The methods of the invention provide a virus like particle assembled from the carrier polypeptides and from the display polypeptides, where the particle comprises one or more species of display polypeptides. In some embodiments of the invention, each particle comprises at least 50 display polypeptides and may comprise at least 120 display polypeptides, or more. In some embodiments, at least two different species of display polypeptides are attached to the carrier. Display polypeptides of interest include, without limitation, antigens of interest for vaccinations, cytokines, single chain antibodies, etc. Display polypeptides are usually greater than 15 amino acids in length, usually greater than 30 amino acids, and may be greater than 50 amino acids in length. Display polypeptides also include polypeptides comprising one or more disulfide bonds.

The components of the VLP are preferably produced by cell-free protein synthesis, where an unnatural amino acid is introduced at a selected site, which may be a single selected site, at a position on the exterior surface of the carrier polypeptide, and a position, which may be a single position, on the display polypeptide. The unnatural amino acid may be positioned on the display protein at any site that does not interfere with the desired activity of the protein, usually at a site other than the amino terminus.

The compositions of the invention are useful for various purposes where it is desirable to efficiently and directly attach multiple polypeptides to a single carrier entity, particularly where two or more different polypeptides are attached to a single carrier. In some embodiments the compositions are used for immunization, e.g. where the display is antigen or antibody in combination with one or more cytokines. In other embodiments the VLP provides a nanostructure template. In other embodiments the VLP provides a carrier for drug or gene delivery where the display is a targeting polypeptide.

### BRIEF DESCRIPTION OF THE DRAWINGS

FIG. 1: SDS-Page gel (10% Bis-Tris Gel w/MES running buffer, Invitrogen; 60 min at 60 mA running conditions; SimplySafe Stain, Invitrogen) and autoradiogram of the same gel after a 1 day exposure. Lanes: M. Mark 12 protein ladder, 1. Positive control of CFPS product after producing the wild type MS2 coat protein from the plasmid pET24a-MS2 cp which does not contain the amber stop codon for incorporation of an unnatural amino acid, 2. CFPS product after producing MS2 coat protein with p-azido-L-phenylalanine incorporated at residue #15, 3. CFPS product after producing MS2 coat protein with p-propargyloxyphenylalanine incorporated at residue #15, 4. Negative control in which CFPS occurred under the same conditions as lane 3 except mutated Methanococcus jannaschii tRNA synthetase was not added, 5. Autoradiogram of lane 1, 6. Autoradiogram of lane 2, 7. Autoradiogram of lane 3, 8. Autoradiogram of lane 4.

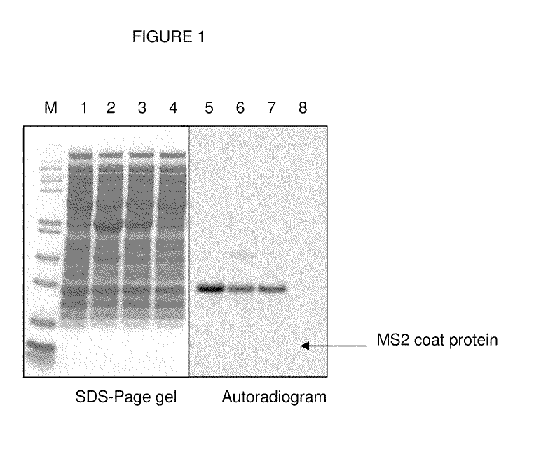

FIG. 2: Profile of the μg of unnatural amino acid containing MS2 coat protein per fraction of sucrose gradient after ultracentrifugation as determined by scintillation counting the radiolabeled protein. Assembled VLP is in fractions 9 through 15 (Bundy 2008).

FIG. 3: SDS-Page gel (10% Bis-Tris Gel w/MOPS running buffer, Invitrogen; 60 min at 60 mA running conditions; SimplySafe Stain, Invitrogen) and autoradiogram of the same gel after a 1 day exposure and overexposed. Left Lanes: M. Mark 12 protein ladder, 1. Click reaction product of 1 equivalent pPaMS2 and 2 equivalents AzmGMCSF 2. Click reaction product of 1 equivalent pPaMS2 and 2 equivalents AzIM9-scFv, 3. Click reaction product of 1 equivalent pPaMS2, 1 equivalent AzmGMCSF, and 1 equivalent AzIM9-scFv, 4. Negative control Click reaction product of 1 equivalent pPaMS2, 1 equivalent AzmGMCSF, and 1 equivalent AzIM9-scFv which was treated the same as Lane 3 except the Cu(I) catalyst was not added, M. Mark 12 protein ladder. Middle Autoradiogram of left SDS-Page gel after 1 day exposure. Lanes 1 through 4 correspond to lanes 1 through 4 of the SDS-Page gel. Right Overexposed autoradiogram of left SDS-Page gel. Lanes 1 through 4 correspond to lanes 1 through 4 of the SDS-Page gel. Expected location of the protein and protein complexed also shown by arrows as estimated from protein and protein complexes molecular weights referenced to the marker. pPa=p-propargyloxyphenylalanine, Az=p-azido-L-phenylalanine.

### DETAILED DESCRIPTION OF THE EMBODIMENTS

Compositions and methods are provided for the control of direct protein attachment to virus like particles. In the methods of the invention, virus structural proteins that have been modified to comprise an unnatural amino acid and have assembled into a carrier VLP are reacted with one or more “display” polypeptides that also comprise an unnatural amino acid. The unnatural amino acid on the virus structural protein, or “carrier”, is different from, and reactive with, the unnatural amino acid present on the display polypeptide(s).

In some embodiments of the invention two, three or more different display polypeptides are attached to the carrier VLP. Each of the display proteins comprises an unnatural amino acid that is different than the unnatural amino acid present on the carrier protein, and which may be the same or different from the unnatural amino acid present on other display proteins. The unnatural amino acid is normally positioned at an accessible site other than the amino terminus. Display polypeptides of interest are usually greater than 15 amino acids in length, and may be greater than 30 amino acids, greater than 50 amino acids, or more.

In some embodiments of the invention, the first unnatural amino acid comprises a reactive alkyne moiety, including, without limitation, p-propargyloxyphenylalanine; and the second unnatural amino acid comprises a reactive azide moiety, including, without limitation, p-azido-L-phenylalanine. Those of skill in the art will appreciate that the first unnatural amino acid may be synthesized into either the display or the carrier polypeptide; where the second unnatural amino acid is then included in the complementary polypeptide.

In some embodiments of the invention, the carrier protein comprises an unnatural amino acid at a single position. In some embodiments of the invention, the carrier protein comprises an unnatural amino acid at not more than three, usually not more than two, different positions. In some embodiments of the invention, each display protein comprises an unnatural amino acid at a single position. In some embodiments of the invention, each display protein comprises an unnatural amino acid at not more than three, usually not more than two, different positions.

High yield production of the display modified virus like particles (VLP) is accomplished by synthesis of the carrier polypeptides and the display polypeptides in separate prokaryotic cell-free reactions comprising at least one orthogonal tRNA aminoacylated with an unnatural amino acid, where the orthogonal tRNA base pairs with a nonsense codon that is not normally associated with an amino acid, e.g. a stop codon; a 4 by codon, etc. Included in the methods are combined transcription-translation reactions. The invention uses nonsense codon suppression during cell-free protein synthesis to produce high yields of polypeptides containing unnatural amino acids. Polypeptides of interest include, without limitation, proteins containing disulfide bonds, any heterogeneous or homogeneous combination of proteins, including fusion proteins, viral coat proteins, and/or proteins originally secreted through or within a cellular membrane.

Unnatural amino acids constitute any amino acid analog that can be used for targeted post-translational modification, particularly where the amino acid analogs present in the display and the carrier polypeptides are directly reactive with each other, e.g. azide and alkyne containing amino acid analogs. The reaction mixture comprises cell extracts, which are optionally amino acid stabilized, reductase minimized, and/or protease mutated cell extracts. The synthesis may be accomplished in combined transcription and translation reactions where the virus coat protein gene may be provided in a suitable vector, e.g. plasmid, etc., operably linked to a promoter active in the transcription system.

In some embodiments, the cell-free reaction mixture will have activation of oxidative phosphorylation as obtained by a combination of reaction conditions, which conditions may include, without limitation, the use of biological extracts derived from bacteria grown on a glucose containing medium; an absence of polyethylene glycol; and optimized magnesium concentration. The system does not require the addition of commonly used secondary energy sources, which energy sources typically contain high energy phosphate bonds, such as phosphoenolpyruvate, creatine phosphate, acetyl phosphate, glucose-6-phosphate, pyruvate or glycolytic intermediates. The reaction may be further improved by employing ions and compounds in the cell-free reaction mixture that are commonly found in the E. coli cytoplasm.

The virus protein of interest is synthesized in a reaction mixture that allows self-assembly of the capsid structure, e.g. a reaction mixture substantially free of polyethylene glycol. In some embodiments, the VLP is assembled from a single coat protein. In other embodiments, the VLP is assembled from two, three or more coat proteins. Bacteriophage VLP are of particular interest.

The methods of the invention provide for high yields of active, i.e. self-assembling, protein. In some embodiments, the yield of active virus coat protein is at least about 50 μg/ml of reaction mixture; at least about 100 μg/ml of reaction mixture; at least about 250 μg/ml, at least about 400 μg/ml of reaction mixture; or more. A substantial portion of the coat protein is assembled into stable VLPs, usually at least about 25%, at least about 50%, at least about 75% or more.

Following assembly of the VLP, the assembled particles may be separated from free proteins by any convenient method, e.g. sucrose gradients, centrifugation, chromatography, filtration, and the like and if desired, may be concentrated and/or resuspended in a suitable buffer. The display polypeptide or polypeptides may be similarly separated from the synthesis reaction mixture and concentrated and/or resuspended in a suitable buffer. Where two or more display polypeptides are present, the display polypeptides are typically mixed prior to reacting with the carrier VLP at the desired ratio, e.g. 1:1, 1:2, 2:1, 1:3, 3:1, 4:1, 1:4, etc. The ratio of attached display polypeptides to carrier polypeptides and to each other will be determined by the initial ratios of the reactants so that reasonable experimentation will define the initial addition ratios that result in the desired attachment ratios.

In some embodiments of the invention, where the unnatural amino acids comprise reactive azide and alkyne groups, the reaction between display and carrier may by catalyzed with a copper(I) catalyst at a concentration sufficient for catalysis, e.g. at least about 1 μM, at least about 0.1 mM, at least about 1 mM, etc., as is known in the art. The reaction can be performed using commercial sources of copper(I) such as cuprous bromide or iodide or a compound such as tetrakis(acetonitrile)copper(I)hexafluorophosphate as long as the reaction is performed under anaerobic conditions. The reaction can be run in a variety of solvents, and mixtures of water and a variety of (partially) miscible organic solvents including alcohols, DMSO, DMF, tBuOH and acetone work well. The reaction will proceed at room temperature, and is allowed to proceed to the desired level of completion, e.g. at least about 15 minutes, at least about one hour, at least about 4 hours, at least about 8 hours, or more.

The methods of the invention provide for high efficiency of coupling. In some embodiments, usually at least about 50% of the available sites present on the carrier polypeptides are coupled to display polypeptides, and in some embodiments at least about 75%, at least about 80%, at least about 85%, at least about 90% or more of the available sites are coupled.

The display-comprising VLP may be purified, concentrated, dialyzed into suitable buffer, and the like, as is known in the art. The remaining uncoupled unnatural (reactive) amino acids are optionally pegylated to reduce antigenicity and undesirable side reactions.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

The terms “desired protein” or “selected protein” are used interchangeably and refer generally to any peptide or protein having more than about 15 amino acids, which comprises at least one unnatural amino acid, which unnatural amino acid is encoded at a specific site in a protein coding polynucleotide. The polypeptides may be homologous to, or may be exogenous, meaning that they are heterologous, i.e., foreign, to the bacteria from which the bacterial cell-free extract is derived, such as a human protein, viral protein, yeast protein, etc. produced in the bacterial cell-free extract.

Examples of polypeptides suitable as display include, but are not limited to, antigenic proteins such as tumor antigens, viral proteins, bacterial proteins, including tuberculosis antigens, protozoan proteins, including malarial proteins, renin; growth hormones, including human growth hormone; bovine growth hormone; growth hormone releasing factor; parathyroid hormone; thyroid stimulating hormone; lipoproteins; alpha-1-antitrypsin; insulin A-chain; insulin; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES and other chemokines; human macrophage inflammatory protein (MIP-1α); a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as αFGF and βFGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-18; superoxide dismutase; T-cell receptors; surface membrane proteins; decay accelerating factor; viral antigen such as, for example, a portion of the AIDS envelope; transport proteins; homing receptors; addressins; regulatory proteins; antibodies particularly single chain Fv antibodies; and fragments of any of the above-listed polypeptides.

Virus coat proteins of interest as display or as carrier polypeptides include any of the known virus types, e.g. dsDNA viruses, such as smallpox (variola); vaccinia; herpes viruses including varicella-zoster; HSV1, HSV2, KSVH, CMV, EBV; adenovirus; hepatitis B virus; SV40; T even phages such as T4 phage, T2 phage; lambda phage; etc. Single stranded DNA viruses include phiX-174; adeno-associated virus, etc. Negative-stranded RNA viruses include measles virus; mumps virus; respiratory syncytial virus (RSV); parainfluenza viruses (PIV); metapneumovirus; rabies virus; Ebola virus; influenza virus; etc. Positive-stranded RNA viruses include polioviruses; rhinoviruses; coronaviruses; rubella; yellow fever virus; West Nile virus; dengue fever viruses; equine encephalitis viruses; hepatitis A and hepatitis C viruses; tobacco mosaic virus (TMV); etc. Double-stranded RNA viruses include reovirus; etc. Retroviruses include rous sarcoma virus; lentivirus such as HIV-1 and HIV-2; etc.

Bacteriophage are of particular interest as carrier proteins, e.g. the MS2 bacteriophage. Myoviridae (phages with contractile tails) include mu-like viruses; P1-like viruses, e.g. P1; phiW39, etc.; P2-like viruses; SPO-1-like viruses; T4-like viruses; etc. Podoviridae (phages with short tails) include N4-like viruses; P22-like viruses, e.g. P22; phi-29-like viruses, e.g. phi-29; T7-like viruses, e.g. T3; T7; W31; etc. Siphoviridae (phages with long non-contractile tails) include c2-like viruses; L5-like viruses; Lambda-like viruses, e.g. phage lambda, HK022; HK97, etc.; N15-like viruses; PhiC31-like viruses; psiM1-like viruses; T1-like viruses, e.g. phage T1, etc. Microviridae (isometric ssDNA phages) include Chlamydiamicrovirus; Microvirus, e.g. phage alpha 3, phage WA13, etc.; phage G4; phage phiX174 and related coliphages. Many additional phages known to those of skill in the art remain unclassified. The sequence of many coat proteins are publicly available.

Virus like particle. As used herein, the term “virus like particle” refers to a stable macromolecular assembly of one or more virus proteins, usually viral coat proteins. The number of separate protein chains in a VLP will usually be at least about 60 proteins, about 80 proteins, at least about 120 proteins, or more, depending on the specific viral geometry. In the methods of the invention, the cell-free synthesis reaction mixture provides conditions permissive for self-assembly into the capsid structure, even where the concentration of coat proteins may be dilute relative to the concentrations associated with in vivo viral synthesis, e.g. less than about 500 μg/ml, less than about 400 μg/ml, less than about 250 μg/ml. The methods of the invention provide for synthesis of the coat protein in the absence of the virus polynucleotide genome, and thus the capsid may be empty, or contain non-viral components, e.g. mRNA fragments, etc. The cell-free synthesis reaction mixtures of the present invention surprisingly provide conditions permissive for self-assembly of coat proteins into a capsid structure displaying helical or icosahedral symmetry.

A stable VLP maintains the association of proteins in a capsid structure under physiological conditions for extended periods of time, e.g. for at least about 24 hrs, at least about 1 week, at least about 1 month, or more. Once assembled, the VLP can have a stability commensurate with the native virus particle, e.g. upon exposure to pH changes, heat, freezing, ionic changes, etc. Additional components of VLPs, as known in the art, can be included within or disposed on the VLP. VLPs do not contain intact viral nucleic acids, and they are non-infectious. In some embodiments there is sufficient viral surface envelope glycoprotein and/or adjuvant molecules on the surface of the VLP so that when a VLP preparation is formulated into an immunogenic composition and administered to an animal or human, an immune response (cell-mediated or humoral) is raised.

Viruses can be classified into those with helical symmetry or icosahedral symmetry. Generally recognized capsid morphologies include: icosahedral (including icosahedral proper, isometric, quasi-isometric, and geminate or “twinned”), polyhedral (including spherical, ovoid, and lemon-shaped), bacilliform (including rhabdo- or bullet-shaped, and fusiform or cigar-shaped), and helical (including rod, cylindrical, and filamentous); any of which may be tailed and/or may contain surface projections, such as spikes or knobs.

In one embodiment of the invention, the coat protein is selected from the capsids of viruses classified as having any icosahedral morphology, and the VLP has an icosahedral geometry. Generally, viral capsids of icosahedral viruses are composed of numerous protein sub-units arranged in icosahedral (cubic) symmetry. Native icosahedral capsids can be built up, for example, with 3 subunits forming each triangular face of a capsid, resulting in 60 subunits forming a complete capsid. A representative of this small viral structure is bacteriophage ØX174. Many icosahedral virus capsids contain more than 60 subunits. Many capsids of icosahedral viruses contain an antiparallel, eight-stranded beta-barrel folding motif. The motif has a wedge-shaped block with four beta strands (designated BIDG) on one side and four (designated CHEF) on the other. There are also two conserved alpha-helices (designated A and B), one is between betaC and betaD, the other between betaE and betaF.

Virus coat proteins of interest include any of the known virus type, e.g. dsDNA viruses, such as smallpox (variola); vaccinia; herpes viruses including varicella-zoster; HSV1, HSV2, KSVH, CMV, EBV; adenovirus; hepatitis B virus; SV40; T even phages such as T4 phage, T2 phage; lambda phage; etc. Single stranded DNA viruses include phiX-174; adeno-associated virus, etc. Negative-stranded RNA viruses include measles virus; mumps virus; respiratory syncytial virus (RSV); parainfluenza viruses (PIV); metapneumovirus; rabies virus; Ebola virus; influenza virus; etc. Positive-stranded RNA viruses include polioviruses; rhinoviruses; coronaviruses; rubella; yellow fever virus; West Nile virus; dengue fever viruses; equine encephalitis viruses; hepatitis A and hepatitis C viruses; tobacco mosaic virus (TMV); etc. Double-stranded RNA viruses include reovirus; etc. Retroviruses include rous sarcoma virus; lentiviruses such as HIV-1 and HIV-2; etc.

Bacteriophages are of interest, e.g. the MS2 bacteriophage. Myoviridae (phages with contractile tails) include mu-like viruses; P1-like viruses, e.g. P1; phiW39, etc.; P2-like viruses; SPO-1-like viruses; T4-like viruses; etc. Podoviridae (phages with short tails) include N4-like viruses; P22-like viruses, e.g. P22; phi-29-like viruses, e.g. phi-29; T7-like viruses, e.g. T3; T7; W31; etc. Siphoviridae (phages with long non-contractile tails) include c2-like viruses; L5-like viruses; Lambda-like viruses, e.g. phage lambda, HK022; HK97, etc.; N15-like viruses; PhiC31-like viruses; psiM1-like viruses; T1-like viruses, e.g. phage T1, etc. Microviridae (isometric ssDNA phages) include Chlamydiamicrovirus; Microvirus, e.g. phage alpha 3, phage WA13, etc.; phage G4; phage phiX174 and related coliphages. Many additional phages known to those of skill in the art remain unclassified. The sequence of many coat proteins are publicly available.

The nucleic acid sequence encoding the viral capsid or proteins can be modified to alter the formation of VLPs (see e.g. Brumfield, et al. (2004) J. Gen. Virol. 85: 1049-1053). For example, three general classes of modification are most typically generated for modifying VLP expression and assembly. These modifications are designed to alter the interior, exterior or the interface between adjacent subunits in the assembled protein cage. To accomplish this, mutagenic primers can be used to: (i) alter the interior surface charge of the viral nucleic acid binding region by replacing basic residues (e.g. K, R) in the N terminus with acidic glutamic acids (Douglas et al., 2002b); (ii) delete interior residues from the N terminus (in CCMV, usually residues 4-37); (iii) insert a cDNA encoding an 11 amino acid peptide cell-targeting sequence (Graf et al., 1987) into a surface exposed loop and (iv) modify interactions between viral subunits by altering the metal binding sites (in CCMV, residues 81/148 mutant).

Unnatural amino acids. Examples of unnatural amino acids that can be used in the methods of the invention include: an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analogue containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,α disubstituted amino acid; a β-amino acid; a cyclic amino acid other than proline, etc.

Unnatural amino acids of interest include, without limitation, amino acids that provide a reactant group for CLICK chemistry reactions (see Click Chemistry: Diverse Chemical Function from a Few Good Reactions Hartmuth C. Kolb, M. G. Finn, K. Barry Sharpless Angewandte Chemie International Edition Volume 40, 2001, P. 2004, herein specifically incorporated by reference). For example, the amino acids p-acetyl-L-phenylalanine, p-propargyloxyphenylalanine, and p-azido-L-phenylalanine are of interest.

Orthogonal components. As used herein, orthogonal components include a tRNA aminoacylated with an unnatural amino acid, where the orthogonal tRNA base pairs with a codon that is not normally associated with an amino acid, e.g. a stop codon; a 4 by codon, etc. The reaction mixture may further comprise a tRNA synthetase capable of aminoacylating (with an unnatural amino acid) the cognate orthogonal tRNA. Such components are known in the art, for example as described in U.S. Pat. No. 7,045,337, issued May 16, 2006. The orthogonal tRNA recognizes a selector codon, which may be nonsense codons, such as, stop codons, e.g., amber, ochre, and opal codons; four or more base codons; codons derived from natural or unnatural base pairs and the like. The orthogonal tRNA anticodon loop recognizes the selector codon on the mRNA and incorporates the unnatural amino acid at this site in the polypeptide.

Orthogonal tRNA synthetase is preferably synthesized exogenously, purified and added to the reaction mix of the invention, usually in a defined quantity, of at least about 10 μg/ml, at least about 30 μg/ml, at least about 100 μg/ml, and not more than about 300 μg/ml. The protein may be synthesized in bacterial or eukaryotic cells and purified, e.g. by affinity chromatography, PAGE, gel exclusion chromatography, reverse phase chromatography, and the like, as known in the art.

The orthogonal tRNA may be synthesized in the cells from which the extract for cell-free synthesis is obtained; may be exogenously synthesized, purified and added to the reaction mix, or may be synthesized de novo, where the cell-free synthesis reaction allows for transcription and translation reactions. Where the orthogonal tRNA is synthesized in the cells from which the extract for cell-free synthesis is obtained, the expression may be controlled through appropriate selection of promoters, medium, and the like.

In vitro synthesis, as used herein, refers to the cell-free synthesis of polypeptides in a reaction mix comprising biological extracts and/or defined reagents. The reaction mix will comprise a template for production of the macromolecule, e.g. DNA, mRNA, etc.; monomers for the macromolecule to be synthesized, e.g. amino acids, nucleotides, etc., and such co-factors, enzymes and other reagents that are necessary for the synthesis, e.g. ribosomes, tRNA, polymerases, transcriptional factors, etc. Such synthetic reaction systems are well-known in the art, and have been described in the literature. The cell free synthesis reaction may be performed as batch, continuous flow, or semi-continuous flow, as known in the art.

In some embodiments of the invention, cell free synthesis is performed in a reaction where oxidative phosphorylation is activated, e.g. the CYTOMIM™ system. The activation of the respiratory chain and oxidative phosphorylation is evidenced by an increase of polypeptide synthesis in the presence of O₂. In reactions where oxidative phosphorylation is activated, the overall polypeptide synthesis in presence of O₂ is reduced by at least about 40% in the presence of a specific electron transport chain inhibitor, such as HQNO, or in the absence of O₂. The reaction chemistry may be as described in international patent application WO 2004/016778, herein incorporated by reference.

The CYTOMIM™ environment for synthesis utilizes cell extracts derived from bacterial cells grown in medium containing glucose and phosphate, where the glucose is present initially at a concentration of at least about 0.25% (weight/volume), more usually at least about 1%; and usually not more than about 4%, more usually not more than about 2%. An example of such media is 2YTPG medium, however one of skill in the art will appreciate that many culture media can be adapted for this purpose, as there are many published media suitable for the growth of bacteria such as E. coli, using both defined and undefined sources of nutrients (see Sambrook, J., E. F. Fritsch, and T. Maniatis. 1989. Molecular Cloning: A Laboratory Manual, 2^{nd} edition. Cold Spring Harbor University Press, Cold Spring Harbor, N.Y. for examples of glucose containing media). Alternatively, the culture may be grown using a protocol in which the glucose is continually fed as required to maintain a high growth rate in either a defined or complex growth medium.

The reaction mixture may be supplemented by the inclusion of vesicles, e.g. an inner membrane vesicle solution. Where provided, such vesicles may comprise from about 0 to about 0.5 volumes, usually from about 0.1 to about 0.4 volumes.

In some embodiments, PEG will be present in not more than trace amounts, for example less than 0.1%, and may be less than 0.01%. Reactions that are substantially free of PEG contain sufficiently low levels of PEG that, for example, oxidative phosphorylation is not PEG-inhibited. The molecules spermidine and putrescine may be used in the place of PEG. Spermine or spermidine is present at a concentration of at least about 0.5 mM, usually at least about 1 mM, preferably about 1.5 mM, and not more than about 2.5 mM. Putrescine is present at a concentration of at least about 0.5 mM, preferably at least about 1 mM, preferably about 1.5 mM, and not more than about 2.5 mM. The spermidine and/or putrescine may be present in the initial cell extract or may be separately added.

The concentration of magnesium in the reaction mixture affects the overall synthesis. Often there is magnesium present in the cell extracts, which may then be adjusted with additional magnesium to optimize the concentration. Sources of magnesium salts useful in such methods are known in the art. In one embodiment of the invention, the source of magnesium is magnesium glutamate. A preferred concentration of magnesium is at least about 5 mM, usually at least about 10 mM, and preferably a least about 12 mM; and at a concentration of not more than about 25 mM, usually not more than about 20 mM. Other changes that may enhance synthesis or reduce cost include the omission of HEPES buffer and phosphoenol pyruvate from the reaction mixture.

The system can be run under aerobic and anaerobic conditions. Oxygen may be supplied, particularly for reactions larger than 15 μl, in order to increase synthesis yields. The headspace of the reaction chamber can be filled with oxygen; oxygen may be infused into the reaction mixture; etc. Oxygen can be supplied continuously or the headspace of the reaction chamber can be refilled during the course of protein expression for longer reaction times. Other electron acceptors, such as nitrate, sulfate, or fumarate may also be supplied in conjunction with preparing cell extracts so that the required enzymes are active in the cell extract.

It is not necessary to add exogenous cofactors for activation of oxidative phosphorylation. Compounds such as nicotinamide adenine dinucleotide (NADH), NAD⁺, or acetyl-coenzyme A may be used to supplement protein synthesis yields but are not required. Addition of oxalic acid, a metabolic inhibitor of phosphoenolpyruvate synthetase (Pps), may be beneficial in increasing protein yields, but is not necessary.

The template for cell-free protein synthesis can be either mRNA or DNA, preferably a combined system continuously generates mRNA from a DNA template with a recognizable promoter. Either an endogenous RNA polymerase is used, or an exogenous phage RNA polymerase, typically T7 or SP6, is added directly to the reaction mixture. Alternatively, mRNA can be continually amplified by inserting the message into a template for QB replicase, an RNA dependent RNA polymerase. Purified mRNA is generally stabilized by chemical modification before it is added to the reaction mixture. Nucleases can be removed from extracts to help stabilize mRNA levels. The template can encode for any particular gene of interest.

Other salts, particularly those that are biologically relevant, such as manganese, may also be added. Potassium is generally present at a concentration of at least about 50 mM, and not more than about 250 mM. Ammonium may be present, usually at a concentration of not more than 200 mM, more usually at a concentration of not more than about 100 mM. Usually, the reaction is maintained in the range of about pH 5-10 and a temperature of about 20°-50° C.; more usually, in the range of about pH 6-9 and a temperature of about 25°-40° C. These ranges may be extended for specific conditions of interest.

Metabolic inhibitors to undesirable enzymatic activity may be added to the reaction mixture. Alternatively, enzymes or factors that are responsible for undesirable activity may be removed directly from the extract or the gene encoding the undesirable enzyme may be inactivated or deleted from the chromosome.

Biological extracts. For the purposes of this invention, biological extracts are any preparation comprising the components required for protein synthesis machinery, usually a bacterial cell extract, wherein such components are capable of expressing a nucleic acid encoding a desired protein. Thus, a bacterial extract comprises components that are capable of translating messenger ribonucleic acid (mRNA) encoding a desired protein, and optionally comprises components that are capable of transcribing DNA encoding a desired protein. Such components include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N¹⁰-formyltetrahydrofolate, formylmethionine-tRNAf^{Met} synthetase, peptidyl transferase, initiation factors such as IF-1, IF-2 and IF-3, elongation factors such as EF-Tu, EF-Ts, and EF-G, release factors such as RF-1, RF-2, and RF-3, and the like.

In a preferred embodiment of the invention, the reaction mixture comprises extracts from bacterial cells, e.g. E. coli S30 extracts, as is known in the art. In some embodiments the bacterial strain is modified such that it endogenously expresses an orthogonal tRNA. For convenience, the organism used as a source of extracts may be referred to as the source organism. Methods for producing active extracts are known in the art, for example they may be found in Pratt (1984), Combined transcription-translation in prokaryotic cell-free systems, p. 179-209, in Hames, B. D. and Higgins, S. J. (ed.), Transcription and Translation: A Practical Approach, IRL Press, New York. Kudlicki et al. (1992) Anal Biochem 206(2):389-93 modify the S30 E. coli cell-free extract by collecting the ribosome fraction from the S30 by ultracentrifugation. Zawada and Swartz Biotechnol Bioeng, 2006. 94(4): p. 618-24 and Liu et al., 2005, Biotechnol Progr 21:460 teach a modified procedure for extract preparation.

The bacterial strain from which the extract is derived may be further modified for the purposes of the invention. In one embodiment, the extract is derived from an E. coli strain deficient in one or more proteins, e.g. strain KC6 (A19ΔtonAΔtnaAΔspeAΔendAΔsdaAΔsdaBΔgshA met⁺), KGK10 (A19ΔspeAΔtnaAΔtonAΔendAΔsdaAΔsdaBΔgshAΔgor met⁺ which can include TrxB-HA, ARG1 (A19ΔtonAΔtnaAΔspeAΔendAΔsdaAΔsdaBΔgshA met⁺ OmpTD83A), ARG2 (A19ΔspeAΔtnaAΔtonAΔendAΔsdaAΔsdaBΔgshAΔgor met⁺ OmpTD83A which can include TrxB-HA); MCJ29 (A19ΔspeAΔtnaAΔompTΔptrCΔdegPΔtonAΔendA met⁺) and the like.

Folding, as used herein, refers to the process of forming the three-dimensional structure of polypeptides and proteins, where interactions between amino acid residues act to stabilize the structure. Non-covalent interactions are important in determining structure, and the effect of membrane contacts with the protein may be important for the correct structure. For naturally occurring proteins and polypeptides or derivatives and variants thereof, the result of proper folding is typically the arrangement that results in optimal biological activity, and can conveniently be monitored by assays for activity, e.g. ligand binding, enzymatic activity, etc.

In some instances, for example where the desired product is of synthetic origin, assays based on biological activity will be less meaningful. The proper folding of such molecules may be determined on the basis of physical properties, energetic considerations, modeling studies, and the like.

The synthesis of membrane-associated protein may be followed by direct isolation of the active, membrane associated forms, i.e. in the absence of refolding or post-translational introduction of membranes. The separation procedure may utilize conditions that maintain membrane integrity, as is known in the art or may use any of several membrane active detergents used to isolate membrane proteins as commonly practiced in the art.

Separation procedures of interest include affinity chromatography. Affinity chromatography makes use of the highly specific binding sites usually present in biological macromolecules, separating molecules on their ability to bind a particular ligand. Covalent bonds attach the ligand to an insoluble, porous support medium in a manner that overtly presents the ligand to the protein sample, thereby using natural biospecific binding of one molecular species to separate and purify a second species from a mixture. Antibodies are commonly used in affinity chromatography. Preferably a microsphere or matrix is used as the support for affinity chromatography. Such supports are known in the art and are commercially available, and include activated supports that can be combined to the linker molecules. For example, Affi-Gel supports, based on agarose or polyacrylamide are low pressure gels suitable for most laboratory-scale purifications with a peristaltic pump or gravity flow elution. Affi-Prep supports, based on a pressure-stable macroporous polymer, are suitable for preparative and process scale applications.

Proteins may also be separated by ion exchange chromatography, and/or concentrated, filtered, dialyzed, etc., using methods known in the art. The methods of the present invention provide for proteins containing unnatural amino acids that have biological activity comparable to the native protein. One may determine the specific activity of a protein in a composition by determining the level of activity in a functional assay, quantitating the amount of protein present in a non-functional assay, e.g. immunostaining, ELISA, quantitation on coomasie or silver stained gel, etc., and determining the ratio of biologically active protein to total protein. Generally, the specific activity as thus defined will be at least about 5% that of the native protein, usually at least about 10% that of the native protein, and may be about 25%, about 50%, about 90% or greater.

### Methods for Synthesis

The synthetic reactions may utilize a large scale reactor, small scale, or may be multiplexed to perform a plurality of simultaneous syntheses. Continuous reactions will use a feed mechanism to introduce a flow of reagents, and may isolate the end-product as part of the process. Batch systems are also of interest, where additional reagents may be introduced to prolong the period of time for active synthesis. A reactor may be run in any mode such as batch, extended batch, semi-batch, semi-continuous, fed-batch and continuous, and which will be selected in accordance with the application purpose.

Strategies for synthesis where at least one unnatural amino acid is introduced into the polypeptide strand during elongation include but are not limited to: (I) addition of exogenous purified orthogonal synthetase, unnatural amino acid, and orthogonal tRNA to the cell-free reaction, (II) addition of exogenous purified orthogonal synthetase and unnatural amino acid to the reaction mixture, but with orthogonal tRNA transcribed during the cell-free reaction, (III) addition of exogenous purified orthogonal synthetase and unnatural amino acid to the reaction mixture, but with orthogonal tRNA synthesized by the cell extract source organism. Preferably the orthogonal components are driven by regulatable promoters, so that synthesis levels can be controlled although other measures may be used such as controlling the level of the relevant DNA templates by addition or specific digestion.

In order to prevent degradation of the orthogonal synthetase, the bacterial strain used to produce extracts may have a deleted or mutated ompT (outer membrane protein T). Where ompT is mutated, it is preferably mutated in such a way that the protease function is inactive, but the chaperone function is still present. Such extracts have decreased levels of synthetase degradation relative to an extract without such a mutation or deletion.

The reaction mixture may also be modified to maintain an oxidizing protein folding environment, for example by supplementing the reaction mix with GSSG at a concentration of from about 0.5 mM to about 10 mM, usually from about 1 mM to about 4 mM; supplementing with GSH at a concentration of from about 0.5 mM to about 10 mM, usually from about 1 mM to about 4 mM. Protein components such as 100 μg/mL DsbC or Skp may also be included. Cell extracts are optionally pretreated with iodoacetamide (IAM).

The reactions may be of any volume, either in a small scale, usually at least about 1 μl and not more than about 15 μl, or in a scaled up reaction, where the reaction volume is at least about 15 μl, usually at least about 50 μl, more usually at least about 100 μl, and may be 500 μl, 1000 μl, or greater. In most cases, individual reactions will not be more than about 10 ml, although multiple reactions can be run in parallel. However, in principle, reactions may be conducted at any scale as long as sufficient oxygen (or other electron acceptor) is supplied when needed.

In addition to the above components such as cell-free extract, genetic template, and amino acids, materials specifically required for protein synthesis may be added to the reaction. These materials include salts, folinic acid, cyclic AMP, inhibitors for protein or nucleic acid degrading enzymes, inhibitors or regulators of protein synthesis, adjusters of oxidation/reduction potential(s), non-denaturing surfactants, buffer components, spermine, spermidine, putrescine, etc.

The salts preferably include potassium, magnesium, and ammonium salts (e.g. of acetic acid or glutamic acid). One or more of such salts may have an alternative amino acid as a counter anion. There is an interdependence among ionic species for optimal concentration. These ionic species are typically optimized with regard to protein production. When changing the concentration of a particular component of the reaction medium, that of another component may be changed accordingly. For example, the concentrations of several components such as nucleotides and energy source compounds may be simultaneously adjusted in accordance with the change in those of other components. Also, the concentration levels of components in the reactor may be varied over time. The adjuster of oxidation/reduction potential may be dithiothreitol, ascorbic acid, glutathione and/or their oxidized forms.

In a semi-continuous operation mode, the outside or outer surface of the membrane is put into contact with predetermined solutions that are cyclically changed in a predetermined order. These solutions contain substrates such as amino acids and nucleotides. At this time, the reactor is operated in dialysis, diafiltration batch or fed-batch mode. A feed solution may be supplied to the reactor through the same membrane or a separate injection unit. Synthesized protein is accumulated in the reactor, and then is isolated and purified according to the usual method for protein purification after completion of the system operation. Vesicles containing the product may also be continuously isolated, for example by affinity adsorption from the reaction mixture either in situ or in a circulation loop as the reaction fluid is pumped past the adsorption matrix.

Where there is a flow of reagents, the direction of liquid flow can be perpendicular and/or tangential to a membrane. Tangential flow is effective for recycling ATP and for preventing membrane plugging and may be superimposed on perpendicular flow. Flow perpendicular to the membrane may be caused or effected by a positive pressure pump or a vacuum suction pump or by applying transmembrane pressure using other methods known in the art. The solution in contact with the outside surface of the membrane may be cyclically changed, and may be in a steady tangential flow with respect to the membrane. The reactor may be stirred internally or externally by proper agitation means.

During protein synthesis in the reactor, the protein isolating means for selectively isolating the desired protein may include a unit packed with particles coated with antibody molecules or other molecules for adsorbing the synthesized, desired protein. Preferably, the protein isolating means comprises two columns for alternating use.

The amount of protein produced in a translation reaction can be measured in various fashions. One method relies on the availability of an assay which measures the activity of the particular protein being translated. An example of an assay for measuring protein activity is a luciferase assay system, or chloramphenical acetyl transferase assay system. These assays measure the amount of functionally active protein produced from the translation reaction. Activity assays will not measure full length protein that is inactive due to improper protein folding or lack of other post translational modifications necessary for protein activity.

Another method of measuring the amount of protein produced in combined in vitro transcription and translation reactions is to perform the reactions using a known quantity of radiolabeled amino acid such as ³⁵S-methionine, ³H-leucine or ¹⁴C-leucine and subsequently measuring the amount of radiolabeled amino acid incorporated into the newly translated protein. Incorporation assays will measure the amount of radiolabeled amino acids in all proteins produced in an in vitro translation reaction including truncated protein products. The radiolabeled protein may be further separated on a protein gel, and by autoradiography confirmed that the product is the proper size and that secondary protein products have not been produced.

Following synthesis, the display and carrier polypeptides are reacted as described above. The resulting display-modified VLP may be resuspended in a physiologically acceptable excipient for use as desired.

Kits for the practice of the subject methods may also be provided. Such kits may include bacterial extracts for protein synthesis and site directed insertion of unnatural amino acids, e.g. containing orthogonal tRNA and/or tRNA synthetase or polynucleotides encoding the same, buffers appropriate for reactions where oxidative phosphorylation is activated, and vesicles.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

### Synthesis of Optimized MS2 Gene

### Materials and Methods

Plasmid Construction. With two-step PCR and custom oligonucleotides (Operon Technologies, USA), a ACG15TAG (amber stop codon) substitution was mutated into the MS2 coat protein sequence using the plasmid pET24a-MS2 cp (Bundy and Swartz 2008 Biotechnol Bioeng 100(1):28-37). The change substitutes a stop codon (TAG) for the natural occurring threonine at amino acid location #15. The mutated sequence was ligated into the pET24a(+) vector (Novagen, USA) at the Nde I and Sal I restriction sites and named pMS2-T15Amb. The vector was transformed into DH5α cells (One Shot MAXX Efficiency DH5α-T1^{R} Competent Cells, Invitrogen) and the plasmid was purified with Qiagen Plasmid Maxi Kit (Qiagen, Valencia, Calif.) for use in cell-free protein synthesis reactions. The sequence is as follows.

The T15STOP substituted MS2 coat protein gene nucleotide sequence was optimized for expression in E. coli cell extracts. This gene was inserted using the underlined NdeI and SalI sites into commercial vector pET24a (Novagen, USA) to produce pET24a_MS2cp_STOP.

CATATGGCCAGCAACTTTACCCAGTTCGTTCTGGTTGACAACGGCGGT [symbol]
GGCGACGTGACGGTGGCGCCGTCTAACTTCGCCAACGGTGTTGCGGA
GTGGATTTCTTCTAATTCTCGCAGCCAAGCCTATAAAGTTACGTGTTCTG
TGCGTCAGTCTAGCGCCCAGAATCGCAAGTACACCATTAAAGTGGAGGTG
CCGAAGGTGGCGACGCAAACCGTGGGTGGTGTGGAGCTGCCAGTTGCGGC
CTGGCGTAGCTATCTGAACATGGAGCTGACGATCCCAATCTTTGCCACCA
ATAGCGACTGCGAACTGATTGTGAAGGCCATGCAGGGTCTGCTGAAGGAT
GGTAACCCGATTCCATCTGCCATTGCCGCGAACTCTGGTATCTACTAATA
AGTCGAC

### Example 2

### Expression of MS2 Coat Protein Containing an Unnatural Amino Acid

PANOx-SP Cell-free Expression System. Modified PANOx-SP cell-free reactions were 30 μL in volume and were incubated at 30° C. for 8 hr in 1.5 ml eppendorf tubes. The reaction includes the following components: 1.2 mM ATP, 0.85 mM each of GTP, UTP, and CTP, 34 μg/mL folinic acid, 171 μg/mL E. coli tRNA mixture, 12 nM pET24aMS2_T15STOP plasmid, 100 μg/mL T7 RNA polymerase, 5 μM [U-¹⁴C]-Leucine, 2 mM each of 20 unlabeled amino acids, 2 mM p-azido-phenylalanine or 2 mM p-propargyloxyphenylalanine (synthesized as described by Dieters 2005), between 150 μg/mL and 5 mg/mL of pure Methanococcus jannaschii mutated tyrosine synthetase mutated for selective recognition of p-azido-L-phenylalanine or p-propargyloxyphenylalanine, 0.33 mM nicotinamide adenine dinucleotide (NAD), 0.27 mM coenzyme A (CoA), 30 mM phosphoenolpyruvate, 1.5 mM spermidine, 1 mM putrescine, 170 mM potassium glutamate, 10 mM ammonium glutamate, 20 mM magnesium glutamate, 2.7 mM sodium oxalate, and 28% v/v of S30 extract prepared as described below. T7 RNA polymerase is prepared from E. coli strain BL21 (pAR1219) as described previously (Jewett and Swartz 2002 Prokaryotic systems for in vitro expression. In: Weiner, M., Lu, Q. (Eds.) Gene Cloning and Expression Technologies. Eaton Publishing, Westborough, Mass., pp. 391-411).

Purification of the Methanococcus jannaschii Orthogonal Tyrosine Synthetase. For this experiment the Methanococcus jannaschii tyrosine-synthetase mutant that specifically incorporates p-azido-phenylalanine or p-propargyloxyphenylalanine was produced and purified. The necessary mutations were published previously as AzPheRS-7 for the azido-phenylalanine mutant and pPR-MjRS-1 for the p-propargyloxyphenylalanine mutant (Chin et al. 2002 J Am Chem Soc 124(31):9026-9027; Dieters and Schultz 2005 Bioorg Med Chem Lett 15:1521-1524). This gene was inserted behind a T7 promoter, a hexahistidine tag was attached to the C-terminus, and the resultant plasmid was transformed into the BL21 DE3 pLys cell strain. The cells were grown in LB media and induced at 0.6 OD with 1 mM IPTG. The cells were harvested at 1.2 to 30D and centrifuged at 4000×g for 30 minutes. The cells were then resuspended and washed in S30 buffer (10 mM Tris-Acetate pH8.2, 14 mM Mg Acetate, 60 mM K Acetate) three times in succession. The cell pellet was then resuspended and the cells were lysed via single pass homogenization. The broken cells were then centrifuged at 16,000×g for 30 min. The supernatant was collected and incubated with DNase for 30 minutes at room temperature. The supernatant was then loaded on a 1 or 5 mL Ni-NTA column, which was equilibrated with 10 mM imidazole, 50 mM phosphate buffer (pH 8.0), and 300 mM NaCl. The column was then washed with 10 mL of 50 mM imidazole in the same buffer and eluted with 250 mM imidazole. The purified products were then concentrated with Amicon Ultra-15 Centrifugal Filter Units (5,000 MWCO) and dialyzed with 7,000 MWCO dialysis tubing against 20% w/v sucrose and 20 mM Potassium Phosphate, pH=7.15.

Production of S30 Extract Containing High Concentrations of Orthogonal tRNA. Extracts were generated with the cell strain KC6 (E. coli A19ΔtonAΔtnaAΔspeAΔendAΔsdaAΔsdaBΔgshA met⁺) harboring the pDule-tRNA plasmid (Goerke and Swartz 2008 Early View Online 25 July). The KC6 cell strain containing the pDuletRNAmj plasmid was grown on defined media at 37° C. and 280 RPM (Zawada and Swartz 2004 Biotechnol Bioeng 89(4):407-415) and grown overnight in a 5 mL inoculum which was used to inoculate a 100 mL inoculum which after 4 hours was used to inoculate 1 Liter liter of medium in a 4 liter shake flask. The fermentation was harvested after 3.5 to 4 hours at 4 to 5.5 OD₆₀₀, and extract was prepared by: First, centrifuging down cells at 4,000×g for 30 min and washing 2× with 4 ml S30 Buffer per gram wet cell pellet and the washed cell pellet was flash frozen with liquid nitrogen and stored at −80° C. (Liu et al. 2005 Biotechnol Prog 21:460-465). Second, the cells were thawed on ice and 1 mL S30 buffer was added per gram of wet cells. Third, the cells were homogenized by single pass homogenization at 17,000 to 25,000 psi. Fourth, the lysate was transferred to 15 mL falcon tubes covered with foil and incubated at 37° C. and 280 RPM for 80 min, aliquoted, and then flash frozen in liquid nitrogen and stored at −80° C. for use in cell-free protein synthesis.

Protein Yield Determination. Total synthesized protein yields were determined by TCA-precipitation and radioactivity measurements in a liquid scintillation counter (LS3801, Beckman Coulter, Inc.). Soluble yields were determined by TCA-precipitation and scintillation counting of the supernatants following sample centrifugation at 25° C. and 15,000 RCF for 15 min. The detailed procedure has been described previously (Jewett and Swartz 2004 Biotechnol Prog 20:102-109.).

SDS-PAGE and Autoradiography. Sample was applied to a NuPAGE 10% Bis-Tris Gel (Invitrogen, La Jolla, Calif.) with Mark12 MW Standard (Invitrogen) molecular weight markers. Gels were stained with SimplySafe Stain (Invitrogen) and dried with a gel dryer, model 583 (Bio-Rad, Richmond, Calif.). An autoradiogram was produced by exposing a Molecular Dynamics Storage Phosphor Screen with the dried gel and then phosphorimaging by a typhoon phosphoimager (GE Healthcare, Piscataway, N.J.).

### Results

Using a 15 μl modified PANOxSP system cell-free reaction as described in the methods with the pMS2-T15Amb plasmid, p-azido-L-phenylalanine, and the Methanococcus jannaschii tyrosine-synthetase mutant that specifically incorporates p-azido-phenylalanine; p-azido-phenylalanine was incorporated into MS2 coat protein at total and soluble yields of 225 μg/ml (std=±32 μg/ml) and 161 μg/ml (std=±8 μg/ml) respectively. Using the same system except substituting p-propargyloxyphenylalanine and using the Methanococcus jannaschii tyrosine-synthetase mutant that specifically incorporates p-propargyloxyphenylalanine for the p-azido-L-phenylalanine counterpart; p-propargyloxyphenylalanine was incorporated into MS2 coat protein at total and soluble yields of 179 μg/ml (std=±32 μg/ml) and 131 μg/ml (std=±7 μg/ml) respectively. To verify the incorporation of p-propargyloxyphenylalanine, we performed additional 15 μl PANOxSP system cell-free reactions that were identical to the reactions incorporating p-propargyloxyphenylalanine into MS2 coat protein, except for the omission of the p-propargyloxyphenylalanine. This resulted in a negligible total yield of 2.3 μg/ml (std=±0.5 μg/ml).

### Example 3

### Demonstrating Assembly of MS2 VLP with 180 Accessible p-Azido-Phenylalanines or p-propargyloxyphenylalanines

Dialysis. To remove unincorporated I-[U-⁴C] leucine, the cell-free product (produced as described in example 2 but produced using a 1 mL thin film reaction instead of the 15 μl scale) was dialyzed in 6-8000 MWCO Specra/Pro Molecularporous Membrane Tubing (Spectrum Labs) against 300 mL 10 mM Bis-Tris-HCl, 375 mM sodium chloride, 5 mM ethylenediaminetetraacetic acid, pH between 5.8 and 6.8) overnight with 2 buffer exchanges.

Velocity Sedimentation Analysis. The dialyzed cell-free reaction product was layered on top of a linear gradient of sucrose ranging from 10% to 40% w/v sucrose and centrifuged at 31,000 rpm in a Beckman-Coulter SW-32 swinging bucket rotor (Fullerton, Calif.) in a Beckman L8-M ultracentrifuge at 4° C. for 3.5 hr with “slow” acceleration and deceleration (profile 7). 0.5 mL fractions were collected using a Teledyne Isco Foxy Jr. Density Gradient Fractionation System (Lincoln, Nebr.), and the MS2 coat protein concentration in each fraction was determined by spotting 25 uL on filter paper and measuring radioactivity using a liquid scintillation counter (LS3801, Beckman Coulter, Inc.)

VLP Concentration. Sucrose gradient factions containing VLPs were concentrated by filling Amicon Ultra-4 5,000 MWCO Centrifugal Filter Devices with gradient fractions and TSM buffer to 4 mL. The units were centrifuged for 15 min at 5,000 rpm and 4° C. in a Sorvall RCSB Centrifuge with a Fiberlite F13-14x15cy rotor (Piramoon Tech.) and Fiberlight 15 mL adaptors (Piramoon). The concentrated sample was washed 3× with 10 mM Potassium Phosphate at pH=8.0 and stored at 4° C.

### Results

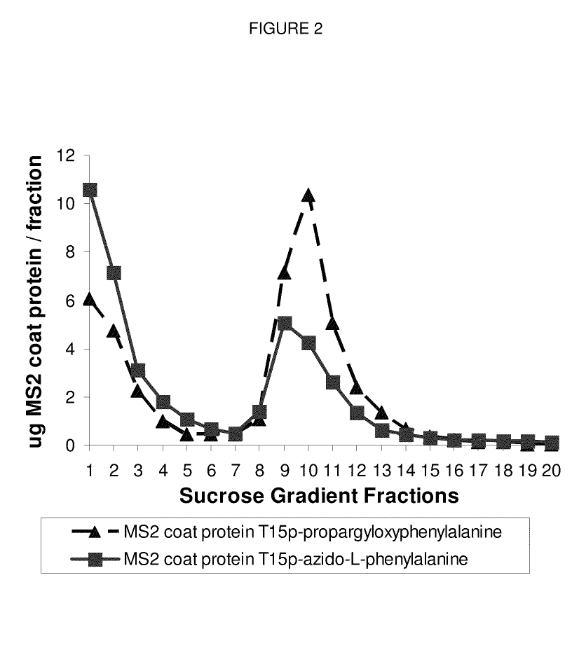

The assembly efficiency of the MS2 coat protein was determined by using a simple material balance after using scintillation counting to determine the amount of radiation that was loaded onto the sucrose gradient and the amount of radiation in the VLP containing fraction. Assembly efficiency for the p-azido-phenylalanine containing MS2 VLP was 30% and the assembly efficiency for the p-propargylphenylalanine containing MS2 VLP was 70% (FIG. 2).

### Example 4

### Direct Attachment of mGMCSF and IM9-scFv to MS2 VLPs Containing p-propargyloxyphenylalanines

Direct Attachment. mGMCSF and IM9-scFv fusion were prepared using a cell-free protein synthesis system as described above and by Goerke and Swartz (2008) Biotechnology and Bioengineering 99:351-67, such that each protein contained one surface accessible p-azido-phenylalanine as well as a C-terminal StrepII-tag. Both were purified from cell-free protein synthesis reaction mixtures using Strep-tactin Sepharose resin per the manufacturer's instructions (IBA Technology, Göttingen, Germany). Both samples were concentrated using Amicon Ultra-4 10,000 MWCO Centrifugal Filter Devices (Millipore Inc.) and dialyzed into 10 mM potassium phosphate, pH=8 using 6-8000 MWCO Specra/Pro Molecular porous Membrane Tubing (Spectrum Labs). In an oxygen free environment 1) p-propargyloxyphenylalaine-MS2 at 3.7 μM (based on the monomer concentration) was combined with 2 molar equivalents of p-azido-phenylalanine-mGMCSF, 2) p-propargyloxyphenylalaine-MS2 at 3.7 μM was combined with 2 molar equivalents of p-azido-phenylalanine-IM9-scFv, 3) p-propargyloxyphenylalaine-MS2 at 3.7 μM was combined with azido-phenylalanine-mGMCSF and p-azido-phenylalanine-IM9-scFv, both at 1 molar equivalent. Cu(I) in the form of tetrakis(acetonitrile)copper(I)hexafluorophosphate was added to a final concentration of 1 mM and the reaction volume was brought to 20 μl with 10 mM potassium phosphate solution, pH=8.0. The reactions were incubated at room temperature in the dark for 8 hrs and then assayed for covalent attachment using SDS-PAGE electrophoresis and autoradiography as explained previously (FIG. 4). Densitometry was performed on the coomassie stained SDS-PAGE gel image using ImageJ software to determine reaction efficiency.

### Results

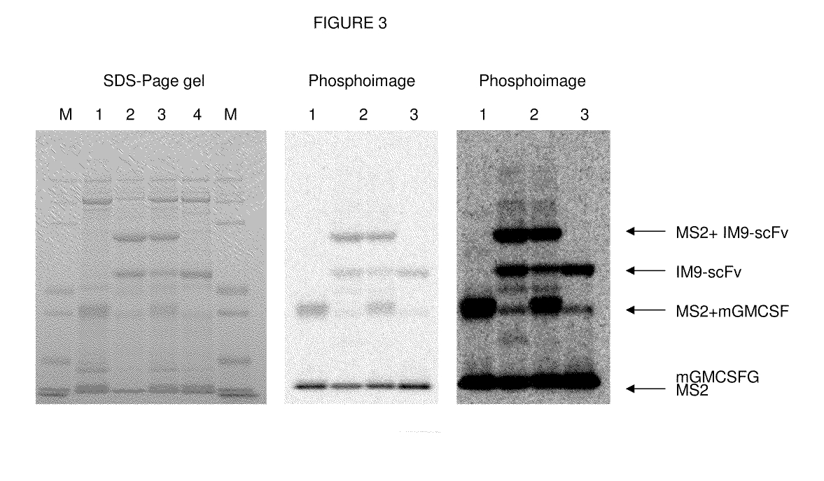

Based on densitometry the number of hGMCSF or/and IM9-scFv attached to the MS2 VLP was determined out of a potential of 180 reactive cites and shown in the Table 1. The coomassie stained SDS-Page gel used to estimate this data is shown in FIG. 3.

Table 1: Based on densitometry of the commassie stained SDS-Page gel, the number of hGMCSF or/and IM9-scFv attached to the MS2 VLP was determined out of a potential of 180 reactive cites.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications, which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

What is claimed is:

1. A method for direct assembly of display polypeptide-modified virus like particles, the method comprising:
combining in a reaction mix: (a) a stable virus like particle (VLP) free of a viral genome comprising carrier virus coat polypeptides modified to comprise at least one first unnatural amino acid at a pre-determined site with (b) display polypeptides modified to comprise at least one second unnatural amino acid, wherein the first unnatural amino acid is different from, and reactive with, the second unnatural amino acid;
reacting the first and second unnatural amino acids in a single step to form a stable attachment;
wherein the VLP comprises at least 60 display polypeptides.

2. The method of claim 1, wherein said carrier virus coat polypeptides comprise less than three unnatural amino acids in each polypeptide.

3. The method of claim 1, wherein said carrier virus coat polypeptides comprise a single unnatural amino acid in each polypeptide.

4. The method of claim 1, wherein said display polypeptides comprise less than three unnatural amino acids in each polypeptide.

5. The method of claim 1, wherein said display polypeptides comprise a single unnatural amino acid in each polypeptide.

6. The method of claim 1, wherein said first and said second unnatural amino acids are selected from p-acetyl-L-phenylalanine, p-propargyloxyphenylalanine, and p-azido-L-phenylalanine.

7. The method of claim 6, wherein said first and said second unnatural amino acids are p-propargyloxyphenylalanine and p-azido-L-phenylalanine.

8. The method of claim 7, wherein said reacting is performed in the presence of a Cu(I) catalyst.

9. The method of claim 1, wherein following said reacting step, at least 50% of the unnatural amino acids present on the carrier virus coat polypeptides are stably attached to display polypeptide.

10. The method of claim 9, wherein following said reacting step, at least 75% of the unnatural amino acids present on the carrier virus coat polypeptides are stably attached to display polypeptide.

11. The method of claim 1, wherein two or more display polypeptides are present in the reaction mix, therein providing for a display polypeptide-modified virus like particle comprising said two or more display polypeptides.

12. The method of claim 1, wherein said display polypeptides are greater than 15 amino acids in length.

13. The method of claim 1, wherein said display polypeptides comprise said second unnatural amino acid at a position other than the amino terminus.

14. A kit for use the method according to claim 1.

15. A display polypeptide-modified virus like particle produced by a method set forth in claim 1.
